(19) United States
(12) Patent Application Publication
(10) Pub. No.: US 2020/0354374 A1
(43) Pub. Date: Nov. 12, 2020

(54) Chemical Entities, Pharmaceutical Formulations, and Methods for Treating Fibrosis

(71) Applicant: Inspira, LLC, Newport Beach, CA (US)

(72) Inventor: Homer L. Pearce, Fairview, TX (US)

(21) Appl. No.: 16/867,557

(22) Filed: May 5, 2020

Related U.S. Application Data
(60) Provisional application No. 62/844,112, filed on May 6, 2019.

(51) Int. Cl. C07D 491/056; C07D 215/54; A61P 11/00

(52) U.S. Cl. CPC C07D 491/056; A61P 11/00; C07D 215/54

(57) ABSTRACT

Disclosed herein are chemical entities, or pharmaceutically acceptable salts thereof, for treating fibrosis, including pulmonary fibrosis, such as idiopathic pulmonary fibrosis. Also disclosed herein are pharmaceutical formulations for treating fibrosis, the pharmaceutical formulations including one or more of the foregoing chemical entities and one or more pharmaceutically acceptable excipients, carriers, vehicles, or a combination thereof. Also disclosed herein are packaged pharmaceutical formulations for treating fibrosis, the packaged pharmaceutical formulations including one of the foregoing pharmaceutical formulations and instructions for using the pharmaceutical formulation to treat a patient having fibrosis or susceptible to fibrosis. Also disclosed herein is a method for treating fibrosis, the method including administering one of the foregoing pharmaceutical formulations.

### PRIORITY

This application claims the benefit of priority to U.S. Provisional Application No. 62/844,112, filed May 6, 2019, which is incorporated by reference in its entirety into this application.

### BACKGROUND

Fibrosis results from formation of an excessive amount of fibrous connective tissue in a tissue or an organ. Progressive fibrosis across organs shares common cellular and molecular pathways involving chronic injury, inflammation and aberrant repair resulting in deposition of extracellular matrix, organ remodeling and ultimately organ failure. Fibrosis is characterized by over expression of transforming growth factorβ (TGFβ) family members and the abnormal and excessive buildup of extracellular matrix (ECM) components, such as fibrillar collagen. This accumulation of ECM triggers progressive organ remodeling and therefore organ dysfunction. Often this fibrotic process is driven by metabolic and inflammatory diseases that result in organ injury and perpetuate the fibrosis. The fact that many different diseases all result in the same fibrotic response in different organs such as the liver, kidney, lung, and skin, speaks for a common disease pathogenesis.

Fibrosis can be defined by the excessive accumulation of fibrous connective tissue (components of the extracellular matrix (ECM) such as collagen and fibronectin) in and around inflamed or damaged tissue, which can lead to permanent scarring, organ malfunction and, ultimately, death, as seen in end-stage liver disease, kidney disease, idiopathic pulmonary fibrosis (IPF) and heart failure. Fibrosis is a pathological feature of most chronic inflammatory diseases. Fibrosis is also a major pathological feature of many chronic autoimmune diseases, including scleroderma, rheumatoid arthritis, Crohn's disease, ulcerative colitis, myelofibrosis, systemic lupus erythematosus and Dupuytren's contracture. Fibrosis also influences tumor invasion and metastasis, chronic graft rejection and the pathogenesis of many progressive myopathies.

Progressive organ fibrosis accounts for one third of all deaths worldwide, yet there are no known curative therapies. Currently, treatments are available for fibrotic disorders including general immunosuppressive drugs such as corticosteroids, and other anti-inflammatory treatments. However, the mechanisms involved in regulation of fibrosis appear to be distinctive from those of inflammation, and anti-inflammatory therapies are seldom effective in reducing or preventing fibrosis. Therefore, a need remains for developing treatments to reduce and prevent fibrosis and control fibrotic disorders.

Exemplary fibrotic diseases and disorders include, but are not limited to, interstitial lung disease, idiopathic pulmonary fibrosis, pneumonia-induced pulmonary interstitial fibrosis, liver cirrhosis, liver fibrosis resulting from chronic hepatitis B or C infection or alcohol, kidney disease, heart disease, and eye diseases including macular degeneration, pemphigoid, and retinal and vitreal retinopathy. Exemplary fibroproliferative disorders include, but are not limited to, systemic and local scleroderma, autoimmune diseases, keloids and hypertrophic scars, atherosclerosis, and restenosis.

An example of a progressive fibrotic disease is pulmonary fibrosis, which includes idiopathic pulmonary fibrosis (IPF), which is a chronic, progressive, and invariably lethal interstitial lung disease of unknown etiology for which there are currently no therapies that stop or reverse the progression of fibrosis. Although a rare and neglected orphan disease, IPF represents the most common cause of death from progressive lung disease with no effective therapy other than lung transplantation. Approximately 50% of people with IPF die within two to three years of the diagnosis (˜17,000 deaths per year in the USA). It is estimated that there are 200,000 IPF patients in the US and North America, and about 34,000 more diagnosed every year. There is estimated to be another 100,000 patients in the 10 most populated European countries. Moreover, the estimated cost of IPF care was $26,378/patient/year in the USA in 2008 and this is before the addition of the cost of Pirfenidone or Nintedanib, costing the US Healthcare System about $4 billion/year. IPF, therefore, represents a major unmet clinical need.

An example of pneumonia-induced pulmonary interstitial fibrosis is that resulting from healing from a viral pneumonia caused by, for example, an adenovirus; any species of Metapneumovirus; a respiratory syndrome-related coronavirus including the severe acute respiratory syndrome coronavirus (SARS-CoV), the severe acute respiratory syndrome coronavirus 2 (SARS-CoV-2 or 2019 novel coronavirus) that causes coronavirus disease 2019 (COVID-19), or the Middle East respiratory syndrome virus (MERS-CoV); any species of Orthohantamovirus; or the like.

Currently there are a large number of pharmaceutical companies working in the fibrosis space, which includes drug discovery for IPF, which is emblematic of drug trials for fibrosis in general. There have been about 13 completed randomized clinical trials within the last decade investigating novel therapies for IPF. All of these compounds were developed based on their success in the bleomycin mouse model of lung fibrosis, which does not mimic clinical aspects of IPF very well. Not surprisingly, the outcomes of these trials have been largely disappointing. Two recent large Phase III clinical trials (Pirfenidone (n=555) and Nintedanib (n=1050)) led to overall reduction in the rate of decline of lung function at early time points, but the compounds did not change overall survival, did not stop ongoing fibrosis, and the slowing of the progression of fibrosis was only modest at best for some patients. It is important to note that the target and mechanism of action of Pirfenidone are not known and that Nintedanib targets many tyrosine kinases. However, these compounds have become standard of care in several countries, due to the lack of any alternative therapy.

Disclosed herein are chemical entities, pharmaceutical formulations, and methods for treating fibrosis.

### SUMMARY

Disclosed herein is a chemical entity of Formula I or a pharmaceutically acceptable salt thereof, wherein R1 is selected from alkyl, alkenyl, alkynyl, cyano, halo, and azido; R2 is selected from heteroarylamino and heterocycloalkylamino; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

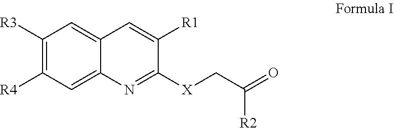

In some embodiments, R1 is selected from methyl, ethyl, ethynyl, cyano, chloro, and azido; R2 is selected from 2-aminobenzothiazole and 2-aminobenzothiophene; R3 and R4 are both methoxy or together form O—CH₂—O; X is selected from S and (CH₂)ₙ; and n is a positive integer from 1 to 3.

In some embodiments, R1 is cyano, R2 is 2-aminobenzothiazole, R3 and R4 together form O—CH₂—O, and X is S, thereby providing the chemical entity of Formula II.

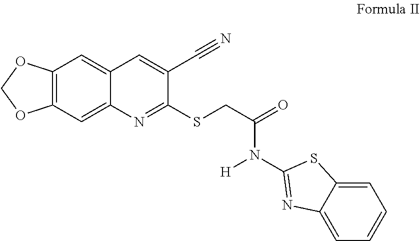 [SMILES: N#Cc1cc2cc3c(cc2nc1SCC(=O)Nc1nc2ccccc2s1)OCO3]

In some embodiments, R1 is ethynyl, R2 is 2-aminobenzothiophene, R3 and R4 together form O—CH₂—O, and X is S, thereby providing the chemical entity of Formula III.

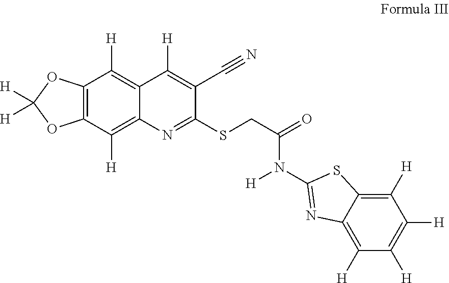 [SMILES: N#Cc1cc2cc3c(cc2nc1SCC(=O)Nc1nc2ccccc2s1)OCO3]

In some embodiments, R1 is ethynyl, R2 is 2-aminobenzothiazole, R3 and R4 together form O—CH₂—O, and X is S, thereby providing the chemical entity of Formula IV.

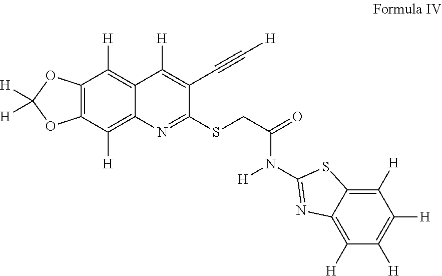 [SMILES: C#Cc1cc2cc3c(cc2nc1SCC(=O)Nc1nc2ccccc2s1)OCO3]

In some embodiments, R1 is methyl, R2 is 2-aminobenzothiazole, R3 and R4 together form O—CH₂—O, and X is S, thereby providing the chemical entity of Formula V.

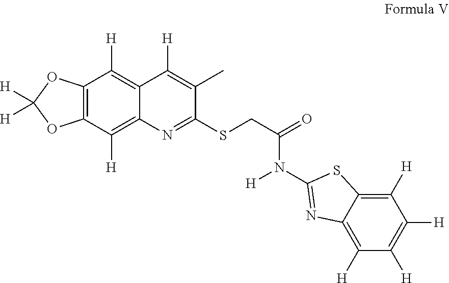 [SMILES: Cc1cc2cc3c(cc2nc1SCC(=O)Nc1nc2ccccc2s1)OCO3]

In some embodiments, R1 is ethyl, R2 is 2-aminobenzothiazole, R3 and R4 together form O—CH₂—O, and X is S, thereby providing the chemical entity of Formula VI.

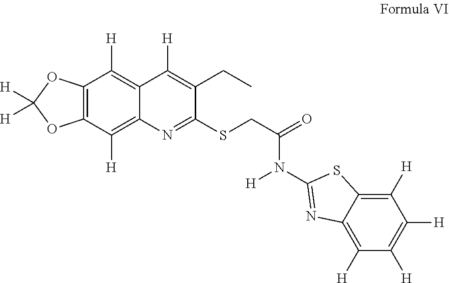 [SMILES: CCc1cc2cc3c(cc2nc1SCC(=O)Nc1nc2ccccc2s1)OCO3]

In some embodiments, R1 is chloro, R2 is 2-aminobenzothiazole, R3 and R4 together form O—CH₂—O, and X is S, thereby providing the chemical entity of Formula VII.

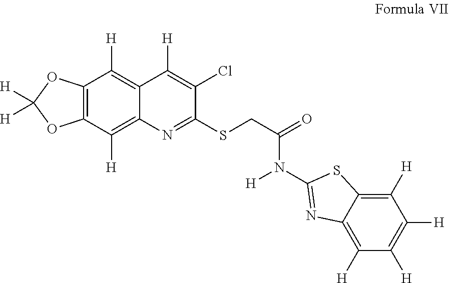 [SMILES: O=C(CSc1nc2cc3c(cc2cc1Cl)OCO3)Nc1nc2ccccc2s1]

In some embodiments, R1 is cyano, R2 is 2-aminobenzothiazole, R3 and R4 together form O—CH₂—O, X is (CH₂)ₙ, and n is 3, thereby providing the chemical entity of Formula VIII.

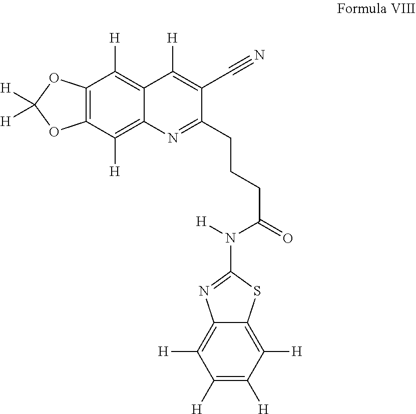 [SMILES: N#Cc1cc2cc3c(cc2nc1CCCC(=O)Nc1nc2ccccc2s1)OCO3]

In some embodiments, R1 is azido, R2 is 2-aminobenzothiazole, R3 and R4 together form O—CH₂—O, X is (CH₂)ₙ, and n is 3, thereby providing the chemical entity of Formula IX.

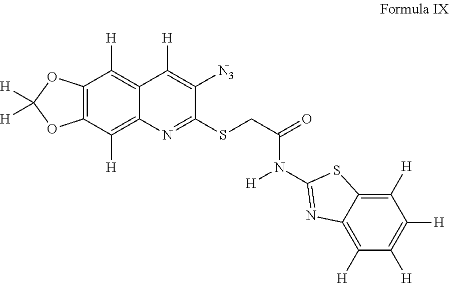 [SMILES: [N-]=[N+]=Nc1cc2cc3c(cc2nc1SCC(=O)Nc1nc2ccccc2s1)OCO3]

Also disclosed herein is a pharmaceutical formulation for treating fibrosis including, in some embodiments a chemical entity of Formula I, or a pharmaceutically acceptable salt thereof, and one or more pharmaceutically acceptable excipients, carriers, vehicles, or a combination thereof. In Formula I, R1 is selected from alkyl, alkenyl, alkynyl, cyano, halo, and azido; R2 is selected from heteroarylamino and heterocycloalkylamino; R3 and R4 are either independently selected from alkoxy and heterocycloalkyl or together form O—CH₂—O; and X is selected from S, O, (CH₂)ₙ, NH, and NR5; R5 is alkyl; and n is a positive integer from 1 to 5.

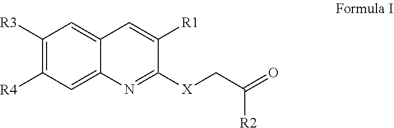

In some embodiments, the pharmaceutical formulation is for treating pulmonary fibrosis.

In some embodiments, the pharmaceutical formulation is for treating idiopathic pulmonary fibrosis.

In some embodiments, the pharmaceutical formulation is for treating viral pneumonia-induced pulmonary fibrosis.

In some embodiments, the viral pneumonia is caused by a virus selected from an adenovirus, a species of Metapneumovirus, a respiratory syndrome-related coronavirus, and any species of Orthohantamovirus.

In some embodiments, the virus is the respiratory syndrome-related coronavirus that causes COVID-19.

In some embodiments, the pharmaceutical formulation is formulated for a mode of administration selected from oral, subcutaneous, transdermal, transmucosal, iontophoretic, intravenous, intraarterial, intramuscular, intraperitoneal, intranasal, subdural, rectal, gastrointestinal, or directly to a specific or affected tissue or organ.

Also disclosed herein is a packaged pharmaceutical formulation for treating fibrosis including, in some embodiments, a pharmaceutical formulation including a chemical entity of Formula I, or a pharmaceutically acceptable salt thereof, and instructions for using the pharmaceutical formulation to treat a patient having fibrosis or susceptible to fibrosis. In Formula I, R1 is selected from alkyl, alkenyl, alkynyl, cyano, halo, and azido; R2 is selected from heteroarylamino and heterocycloalkylamino; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

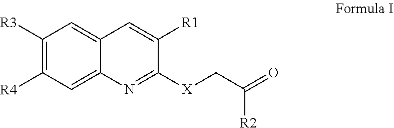

In some embodiments, the pharmaceutical formulation is for treating pulmonary fibrosis and the instructions are for using the pharmaceutical formulation to treat a patient having pulmonary fibrosis or susceptible to pulmonary fibrosis.

In some embodiments, the pharmaceutical formulation is for treating idiopathic pulmonary fibrosis and the instructions are for using the pharmaceutical formulation to treat a patient having idiopathic pulmonary fibrosis or susceptible to idiopathic pulmonary fibrosis.

In some embodiments, the packaged pharmaceutical formulation is for treating viral pneumonia-induced pulmonary fibrosis and the instructions are for using the pharmaceutical formulation to treat a patient having viral pneumonia-induced pulmonary fibrosis or susceptible to viral pneumonia-induced pulmonary fibrosis.

Also disclosed herein is a method for treating fibrosis including, in some embodiments, administering a pharmaceutical formulation including a therapeutically effective amount of a chemical entity of Formula I, or a pharmaceutically acceptable salt thereof. In Formula I, R1 is selected from alkyl, alkenyl, alkynyl, cyano, halo, and azido; R2 is selected from heteroarylamino and heterocycloalkylamino; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

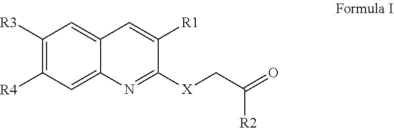

In some embodiments, the fibrosis is pulmonary fibrosis.

In some embodiments, the fibrosis is idiopathic pulmonary fibrosis.

In some embodiments, the fibrosis is viral pneumonia-induced pulmonary fibrosis.

These and other features of the concepts provided herein will become more apparent to those of skill in the art in view of the accompanying drawings and following description, which disclose particular embodiments of such concepts in greater detail.

### DRAWINGS

FIG. 1 provides a ¹H NMR spectrum for the chemical entity of Formula II set forth herein.

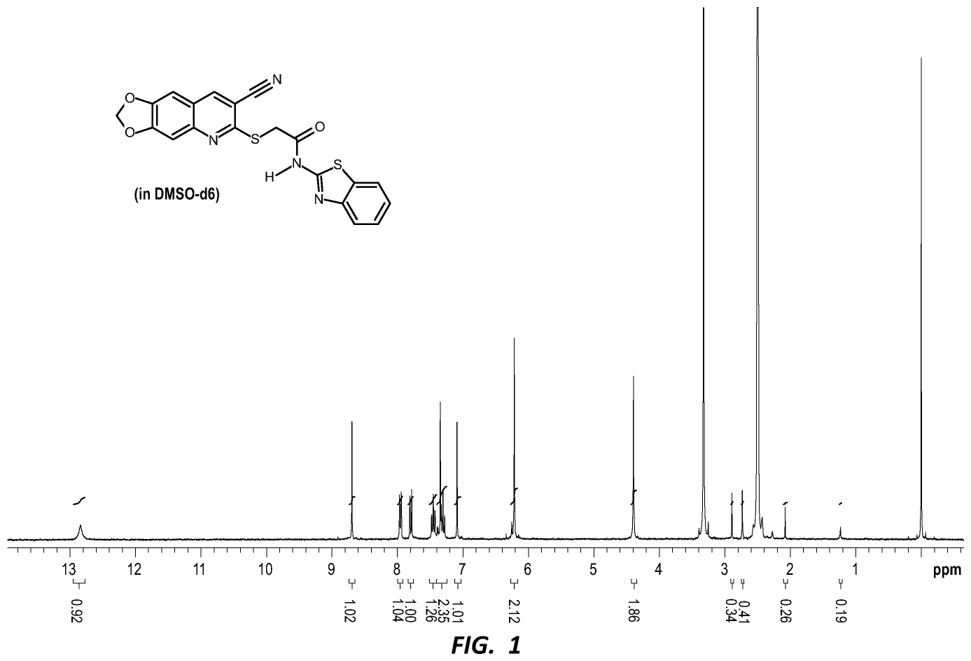

FIG. 2A provides a LCMS liquid chromatogram for the chemical entity of Formula II set forth herein.

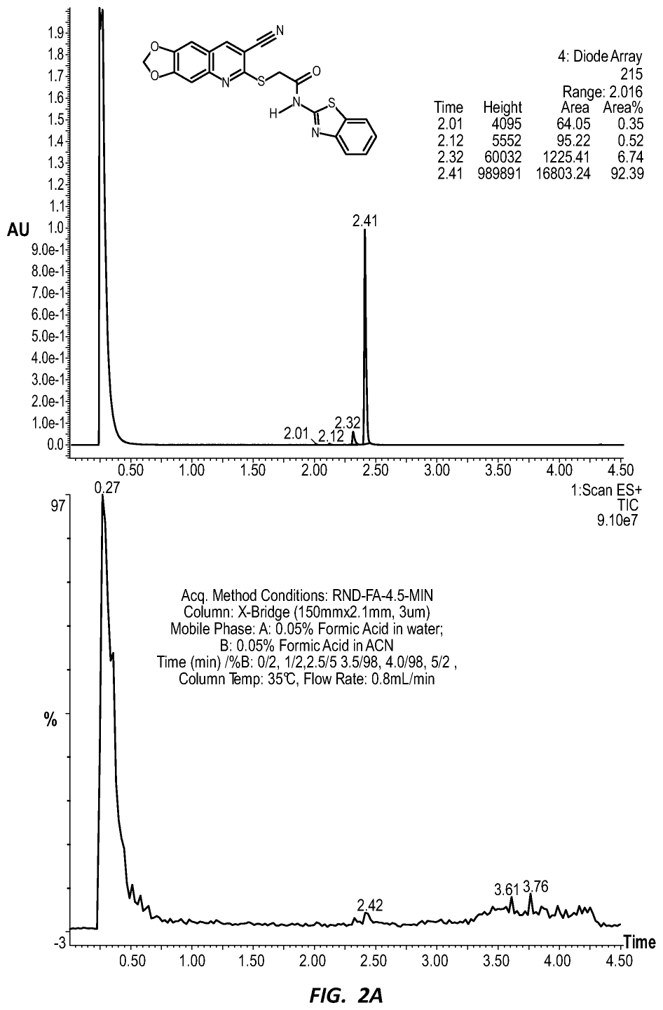

FIG. 2B provides a LCMS mass spectrum for the chemical entity of Formula II set forth herein.

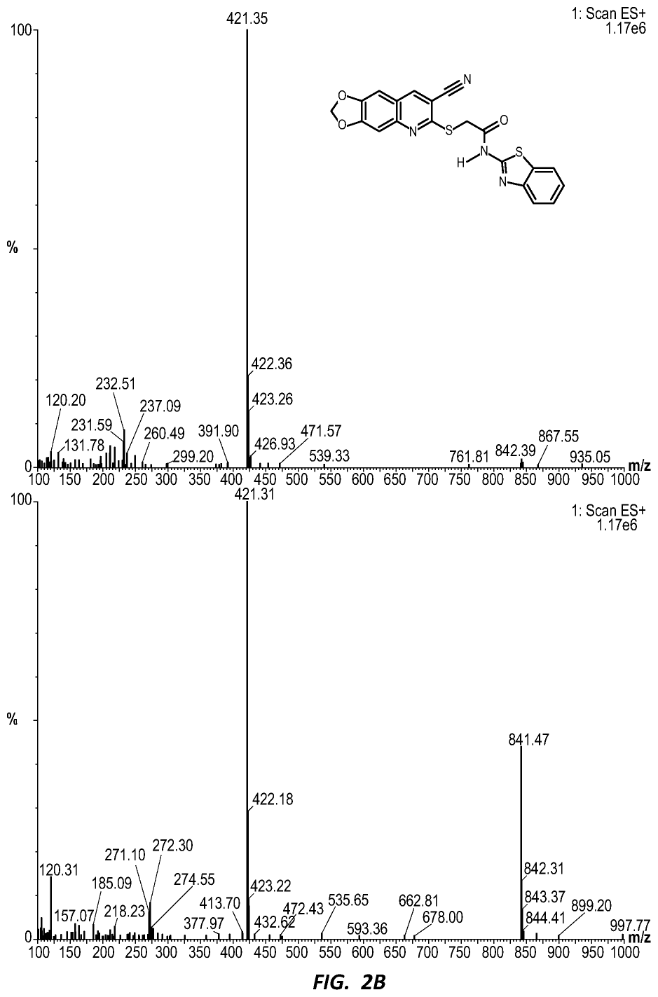

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Singular forms of “a,” “an,” and “the” include plural references unless the context clearly dictates otherwise.

“Optional” or “optionally” indicates a subsequently described event or circumstance occurs or does not occur, and that the description includes instances where the event or circumstance occurs and instances in which it does not. For example, “optionally substituted alkyl” encompasses both “alkyl” and “substituted alkyl” as defined below. It should be understood by those of skill in the art, with respect to any group containing one or more substituents, that such groups are not intended to introduce any substitution or substitution patterns that are sterically impractical, synthetically non-feasible, or inherently unstable.

“Alkyl” encompasses straight chain and branched chain alkyl groups having the indicated number of carbon atoms, usually from 1 to 20 carbon atoms, for example 1 to 8 carbon atoms, such as 1 to 6 carbon atoms. For example, C₁-C₆ alkyl encompasses both straight and branched chain alkyl of from 1 to 6 carbon atoms. When an alkyl residue having a specific number of carbons is named, all branched and straight chain versions having that number of carbons are intended to be encompassed. Thus, for example, “butyl” include n-butyl, sec-butyl, isobutyl, and t-butyl; “propyl” includes n-propyl and isopropyl. “Lower alkyl” refers to alkyl groups having one to seven carbons. In certain embodiments, “lower alkyl” refers to alkyl groups having one to six carbons. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, or the like. Alkylene is a subset of alkyl, referring to the same residues as alkyl, but having two points of attachment. Alkylene groups usually have from 2 to 20 carbon atoms, for example 2 to 8 carbon atoms, such as from 2 to 6 carbon atoms. For example, a C₁ alkylene is a methylene group.

“Alkenyl” refers to an unsaturated branched or straight-chain alkyl group having at least one carbon-carbon double bond derived by the removal of one molecule of hydrogen from adjacent carbon atoms of the parent alkyl. The group can be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; or the like. In certain embodiments, an alkenyl group has from 2 to 20 carbon atoms and in other embodiments, from 2 to 6 carbon atoms. “Lower alkenyl” refers to alkenyl groups having two to six carbons.

“Alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; or the like. In certain embodiments, an alkynyl group has from 2 to 20 carbon atoms and in other embodiments, from 3 to 6 carbon atoms. “Lower alkynyl” refers to alkynyl groups having two to six carbons.

“Cycloalkyl” indicates a non-aromatic carbocyclic ring, usually having from 3 to 7 ring carbon atoms. The ring can be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged ring groups such as norbornane.

The term “alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy, or the like. “Lower alkoxy” refers to alkoxy groups containing one to six carbons.

The term “substituted alkoxy” refers to alkoxy wherein the alkyl constituent is substituted (i.e., —O-(substituted alkyl)) wherein “substituted alkyl” refers to alkyl wherein one or more (such as up to 5, for example, up to 3) hydrogen atoms are replaced by a substituent. A substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

The term “alkoxycarbonyl” refers to a group of the formula (alkoxy)(C═O)-attached through the carbonyl carbon wherein the alkoxy group has the indicated number of carbon atoms. Thus, a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent.

“Acyl” refers to the groups H—C(O)—, (alkyl)-C(O)—, (aryl)-C(O)—, (heteroaryl)-C(O)—, and (heterocycloalkyl)-C(O)—, wherein the group is attached to the parent structure through the carbonyl functionality, and wherein alkyl, aryl, heteroaryl, and heterocycloalkyl are optionally substituted as described herein. Examples include acetyl, benzoyl, propionyl, isobutyryl, t-butoxycarbonyl, benzyloxycarbonyl, or the like. “Lower-acyl” refers to groups containing one to six carbons and “acyloxy” refers to the group O-acyl.

The term “amino” refers to the group —NH₂.

The term “substituted amino” refers to the group —NHR^{a} or —NR^{a}R^{b}, wherein R^{a} and R^{b} are independently chosen from, for example, hydroxy, optionally substituted alkoxy, optionally substituted alkyl, optionally substituted cycloalkyl, optionally substituted acyl, optionally substituted carbamimidoyl, aminocarbonyl, optionally substituted aryl, optionally substituted heteroaryl, optionally substituted heterocycloalkyl, optionally substituted alkoxycarbonyl, sulfinyl, and sulfonyl.

The term “substituted amino” also refers to N-oxides of the groups —NHR^{a}, and NR^{a}R^{b} each as described above. N-oxides can be prepared by treatment of the corresponding amino group with, for example, hydrogen peroxide or m-chloroperoxybenzoic acid.

The term “aminocarbonyl” refers to the group —CONR^{a}R^{b}, wherein R^{a} and R^{b} are independently chosen from, for example, hydrogen, hydroxy, optionally substituted alkoxy, optionally substituted alkyl, optionally substituted cycloalkyl, optionally substituted acyl, optionally substituted carbamimidoyl, aminocarbonyl, optionally substituted aryl, optionally substituted heteroaryl, optionally substituted heterocycloalkyl, optionally substituted alkoxycarbonyl, sulfinyl, and sulfonyl.

“Aryl” encompasses 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene.

For example, aryl includes 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment can be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

“Aralkoxy” refers to the group —O-aralkyl. Similarly, “heteroaralkoxy” refers to the group —O-heteroaralkyl; “aryloxy” refers to —O-aryl; and “heteroaryloxy” refers to the group —O-heteroaryl.

“Aralkyl” refers to a residue in which an aryl moiety is attached to the parent structure via an alkyl residue. Examples include benzyl, phenethyl, phenylvinyl, phenylallyl, or the like. “Heteroaralkyl” refers to a residue in which a heteroaryl moiety is attached to the parent structure by way of an alkyl residue. Examples include furanylmethyl, pyridinylmethyl, pyrimidinyl ethyl, or the like.

“Halogen” or “halo” refers to fluorine, chlorine, bromine, or iodine. Dihaloaryl, dihaloalkyl, trihaloaryl etc. refer to aryl and alkyl substituted with a plurality of halogens, but not necessarily a plurality of the same halogen; thus, 4-chloro-3-fluorophenyl is within the scope of dihaloaryl.

“Heteroaryl” encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring; and tricyclic heterocycloalkyl rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.

For example, heteroaryl includes a 5- to 7-membered heterocycloalkyl, aromatic ring fused to a 5- to 7-membered cycloalkyl or heterocycloalkyl ring. For such fused, bicyclic heteroaryl ring systems, wherein only one of the rings contains one or more heteroatoms, the point of attachment can be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridazinyl, triazolyl, quinolinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl, cycloalkyl, or heterocycloalkyl, as defined herein.

Substituted heteroaryl also includes ring systems substituted with one or more oxide (—O) substituents, such as pyridinyl N-oxides.

By “heterocycloalkyl” is meant a single, non-aromatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. The ring can be saturated or have one or more carbon-carbon double bonds. Suitable heterocycloalkyl groups include, for example (as numbered from the linkage position assigned priority 1), 2-pyrrolidinyl, 2,4-imidazolidinyl, 2,3-pyrazolidinyl, 2-piperidyl, 3-piperidyl, 4-piperidyl, and 2,5-piperizinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (═O) or oxide (—O⁻) substituents, such as piperidinyl N-oxide, morpholinyl-N-oxide, 1-oxo-1-thiomorpholinyl and 1,1-dioxo-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Isomers” are different chemical entities that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The symbol “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a chemical entity is a pure enantiomer the stereochemistry at each chiral carbon can be specified by either R or S. Resolved stereoisomers whose absolute configuration is unknown can be designated (+) or (−) depending on the direction (dextro- or levorotatory) that they rotate plane polarized light at the wavelength of the sodium D line. Certain chemical entities disclosed herein contain one or more asymmetric centers and can thus give rise to enantiomers, diastereomers, and other stereoisomeric forms that can be defined, in terms of absolute stereochemistry, as (R) or (S). All such possible isomers, including racemic mixtures, optically pure forms and intermediate mixtures are include unless specified otherwise. Optically active (R)- and (S)-isomers can be prepared using chiral synthons or chiral reagents, or resolved using conventional techniques. When the chemical entities disclosed herein contain olefinic double bonds or other centers of geometric asymmetry, and unless specified otherwise, it is intended that the chemical entities include both E and Z geometric isomers.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A leaving group or atom is any group or atom that, under the reaction conditions, leaves from the starting material, thus promoting reaction at a specified site. Suitable examples of such groups unless otherwise specified are halogen atoms, mesyloxy, p-nitrobenzensulphonyloxy and tosyloxy groups.

Protecting group has the meaning conventionally associated with it in organic synthesis; that is, a group that selectively blocks one or more reactive sites in a multifunctional chemical entity such that a chemical reaction can be carried out selectively on another unprotected reactive site and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a hydroxy protected form is where at least one of the hydroxy groups present in a chemical entity is protected with a hydroxy protecting group. Likewise, amines and other reactive groups can similarly be protected.

The term “pharmaceutically acceptable salt” refers to salts that retain the biological effectiveness and properties of the chemical entities disclosed herein and, which are not biologically or otherwise undesirable. In many cases, the chemical entities disclosed herein are capable of forming acid or base salts by virtue of the presence of amino or carboxyl groups, or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, or the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, or the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, or the like. Organic bases from which salts can be derived include, for example, primary, secondary, and tertiary amines, substituted amines including naturally occurring substituted amines, cyclic amines, basic ion exchange resins, or the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “solvate” refers to a chemical entity (e.g., a chemical entity selected from Formula I, or a pharmaceutically acceptable salt thereof) in physical association with one or more molecules of a pharmaceutically acceptable solvent. It should be understood that a chemical entity of Formula I encompasses that of Formula I and solvates thereof, as well as mixtures thereof.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl, wherein one or more (such as up to 5, for example, up to 3) hydrogen atoms are replaced by a substituent.

The term “sulfanyl” refers to the groups —S-(optionally substituted alkyl), —S-(optionally substituted cycloalkyl), —S-(optionally substituted aryl), —S-(optionally substituted heteroaryl), and —S-(optionally substituted heterocycloalkyl).

The term “sulfinyl” refers to the groups —S(O)—H, —S(O)-(optionally substituted alkyl), —S(O)-(optionally substituted cycloalkyl), —S(O)-(optionally substituted amino), —S(O)-(optionally substituted aryl), —S(O)-(optionally substituted heteroaryl), and —S(O)-(optionally substituted heterocycloalkyl).

The term “sulfonyl” refers to the groups: —S(O₂)—H, —S(O₂)-(optionally substituted alkyl), —S(O₂)-(optionally substituted cycloalkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

The term “therapeutically effective amount” or “effective amount” refers to that amount of a chemical entity disclosed herein that is sufficient to effect treatment, as defined below, when administered to a subject in need of such treatment. The therapeutically effective amount varies depending upon the subject and disease condition being treated, the weight and age of the subject, the severity of the disease condition, the particular chemical entity disclosed herein, the dosing regimen to be followed, timing of administration, the manner of administration, or the like, all of which can readily be determined by one of ordinary skill in the art.

Unless defined otherwise, all technical and scientific terms used herein have the same meaning as commonly understood by those of ordinary skill in the art.

Fibrosis represents aberrant wound healing that is not able to resolve due to ongoing tissue injury, chronic inflammation, and extracellular matrix deposition. The chemical entities disclosed herein advance the field because they stop the cycle of chronic inflammation, promote a resolutive macrophage phenotype, break down the collagen, and reverse the activated fibroblast phenotype associated with fibrosis. In this way, the chemical entities disclosed herein promote reversal of scarring and normal wound healing.

### Chemical Entities

A chemical entity for treating fibrosis includes a chemical entity of Formula I, or a pharmaceutically acceptable salt thereof, wherein R1 is selected from alkyl, alkenyl, alkynyl, cyano, halo, and azido; R2 is selected from arylamino, cycloalkylamino, heteroarylamino, and heterocycloalkylamino optionally substituted with alkyl, alkenyl, alkynyl, halo, acyl, alkoxycarbonyl, aminocarbonyl, or a combination thereof; R3 and R4 are either independently selected from alkoxy and heterocycloalkyl or together form O—CH₂—O or O—CH₂CH₂—O; X is selected from S, O, (CH₂)ₙ, NH, and NR5; R5 is alkyl; and n is a positive integer from 1 to 5.

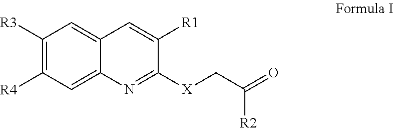

In some embodiments, R1 is selected from alkyl, alkenyl, alkynyl, cyano, halo, and azido; R2 is selected from heteroarylamino and heterocycloalkylamino optionally substituted as set forth above; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

In some embodiments, R1 is selected from methyl, ethyl, ethynyl, cyano, chloro, and azido; R2 is selected from 2-aminobenzothiazole and 2-aminobenzothiophene optionally substituted as set forth above at any one or more carbon atoms of carbon atoms 4, 5, 6, or 7 as conventionally numbered; R3 and R4 are both methoxy or together form O—CH₂—O; X is selected from S and (CH₂)ₙ; and n is a positive integer from 1 to 3.

In some embodiments, R1 is cyano, R2 is 2-aminobenzothiazole, R3 and R4 together form O—CH₂—O, and X is S, thereby providing the chemical entity of Formula II.

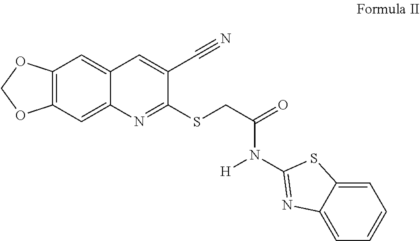 [SMILES: N#Cc1cc2cc3c(cc2nc1SCC(=O)Nc1nc2ccccc2s1)OCO3]

In some embodiments, R1 is ethynyl, R2 is 2-aminobenzothiophene, R3 and R4 together form O—CH₂—O, and X is S, thereby providing the chemical entity of Formula III.

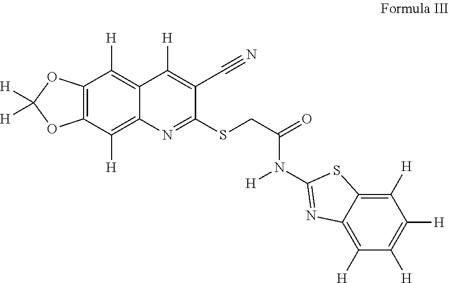 [SMILES: N#Cc1cc2cc3c(cc2nc1SCC(=O)Nc1nc2ccccc2s1)OCO3]

In some embodiments, R1 is ethynyl, R2 is 2-aminobenzothiazole, R3 and R4 together form O—CH₂—O, and X is S, thereby providing the chemical entity of Formula IV.

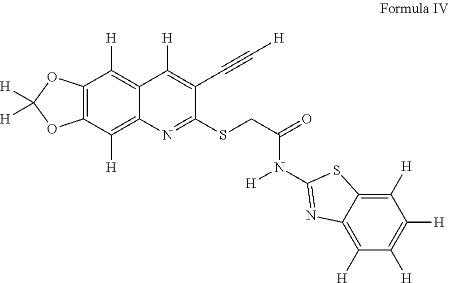 [SMILES: C#Cc1cc2cc3c(cc2nc1SCC(=O)Nc1nc2ccccc2s1)OCO3]

In some embodiments, R1 is methyl, R2 is from 2-aminobenzothiazole, R3 and R4 together form O—CH₂—O, and X is S, thereby providing the chemical entity of Formula V.

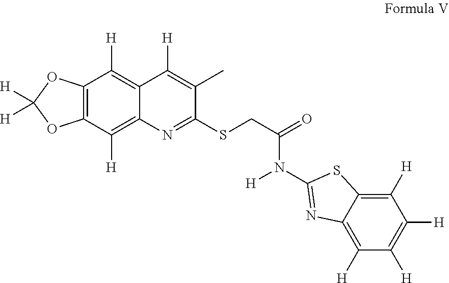 [SMILES: Cc1cc2cc3c(cc2nc1SCC(=O)Nc1nc2ccccc2s1)OCO3]

In some embodiments, R1 is ethyl, R2 is from 2-aminobenzothiazole, R3 and R4 together form O—CH₂—O, and X is S, thereby providing the chemical entity of Formula VI.

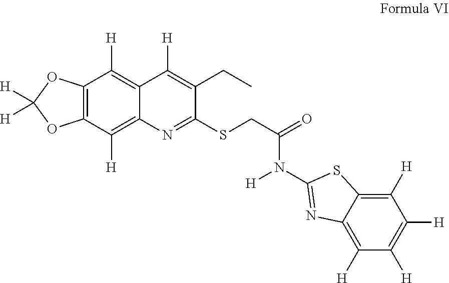 [SMILES: CCc1cc2cc3c(cc2nc1SCC(=O)Nc1nc2ccccc2s1)OCO3]

In some embodiments, R1 is chloro, R2 is 2-aminobenzothiazole, R3 and R4 together form O—CH₂—O, and X is S, thereby providing the chemical entity of Formula VII.

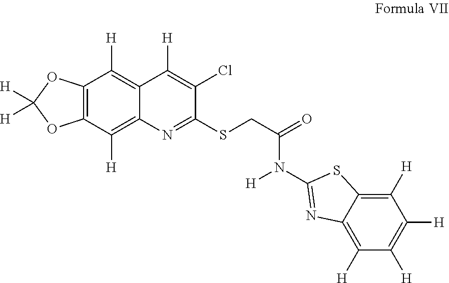 [SMILES: O=C(CSc1nc2cc3c(cc2cc1Cl)OCO3)Nc1nc2ccccc2s1]

In some embodiments, R1 is cyano, R2 is 2-aminobenzothiazole, R3 and R4 together form O—CH₂—O, X is (CH₂)ₙ, and n is 3, thereby providing the chemical entity of Formula VIII.

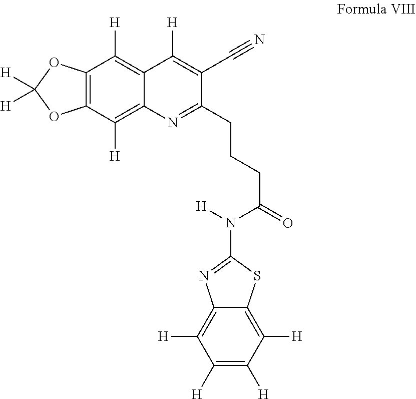 [SMILES: N#Cc1cc2cc3c(cc2nc1CCCC(=O)Nc1nc2ccccc2s1)OCO3]

In some embodiments, R1 is azido, R2 is 2-aminobenzothiazole, R3 and R4 together form O—CH₂—O, X is (CH₂)ₙ, and n is 3, thereby providing the chemical entity of Formula IX. The chemical entity of Formula IX is useful as a photo-affinity probe.

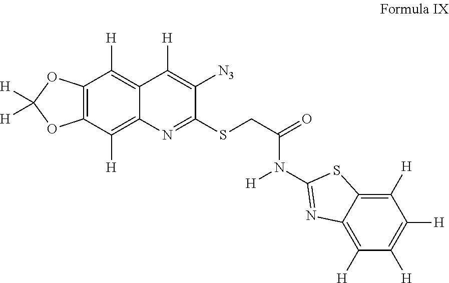 [SMILES: [N-]=[N+]=Nc1cc2cc3c(cc2nc1SCC(=O)Nc1nc2ccccc2s1)OCO3]

In some embodiments, the chemical entity of Formula I, or the pharmaceutically acceptable salt thereof, is selected from the following eight chemical entities:

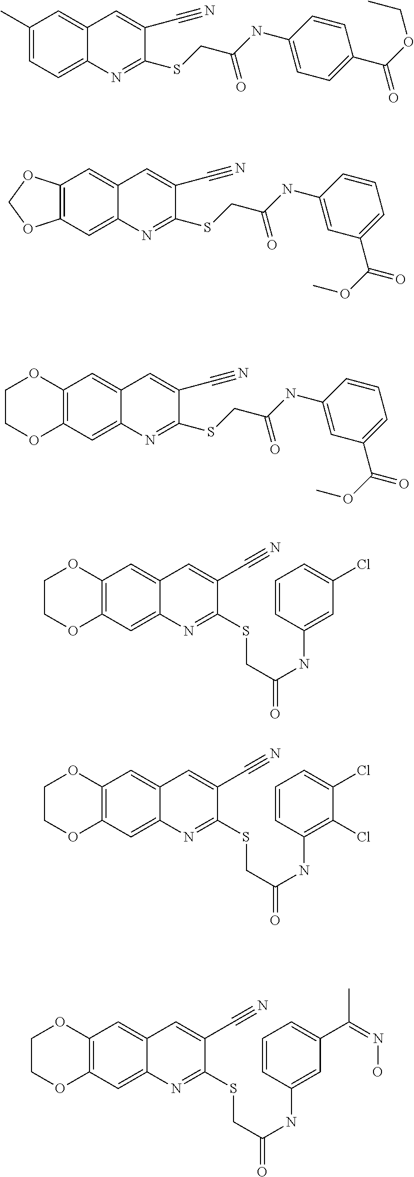

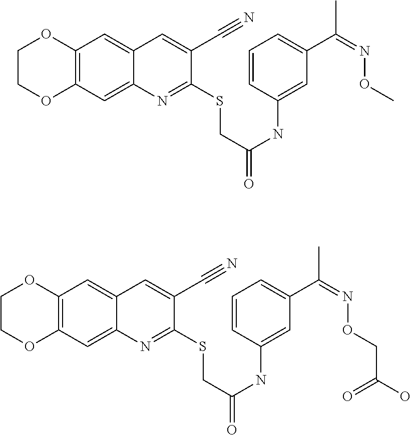 [SMILES: CO/N=C(/C)c1cccc(NC(=O)CSc2nc3cc4c(cc3cc2C#N)OCCO4)c1 ; C/C(=N/OCC(=O)O)c1cccc(NC(=O)CSc2nc3cc4c(cc3cc2C#N)OCCO4)c1]

No chemical entity of the foregoing eight chemical entities is known to have been used in a pharmaceutical formulation for treating fibrosis, a packaged pharmaceutical formulation for treating fibrosis, or a method for treating fibrosis.

In some embodiments, the chemical entity of Formula I, or the pharmaceutically acceptable salt thereof, is for treating pulmonary fibrosis including idiopathic pulmonary fibrosis or viral pneumonia-induced pulmonary fibrosis. The viral pneumonia can be caused by a virus selected from an adenovirus, a species of Metapneumovirus, a respiratory syndrome-related coronavirus, and any species of Orthohantamovirus. For example, the virus can be the respiratory syndrome-related coronavirus that causes COVID-19.

### Preparation of Chemical Entities

The chemical entity of Formula II set forth above can be prepared in accordance with the chemical synthesis of Scheme 1.

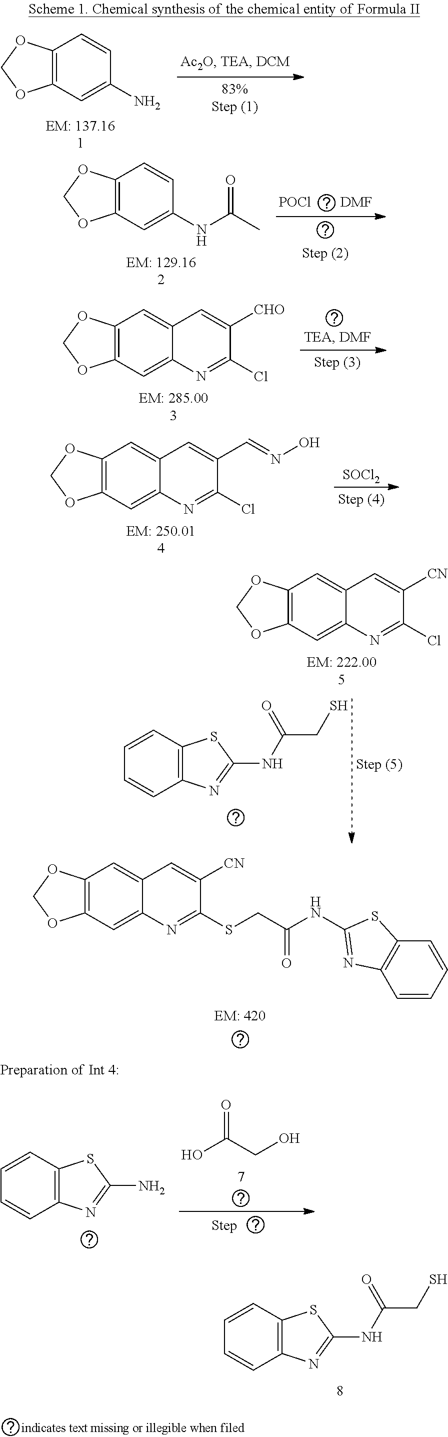

In step 1, for example, to the chemical entity 1 (1 eq) and triethylamine (3 eq) in dry dichloromethane is added acetic anhydride (1.5 eq) at about 0° C. under argon at atmospheric pressure. The resulting reaction mixture is allowed to stir at room temperature for about 3 hours or more. After a standard workup and removal of solvent by evaporation, the crude chemical entity 2 is co-distilled twice with toluene twice (2×5 ml) to provide the chemical entity 2.

In step 2, for example, to the chemical entity 2 (1 eq) in dry dimethylformamide (2.5 eq) is slowly added POCl₃ (7 eq) at 0° C. under argon at atmospheric pressure. The resulting reaction mixture is allowed to stir at room temperature for about 30 minutes. Then, the reaction mixture is heated to about 75° C. for about 16 hours. After a standard workup, the crude the chemical entity 3 is washed with n-pentane to provide the chemical entity 3.

In step 3, for example, to the chemical entity 3 (1 eq) in dry dimethylformamide is added hydroxylamine HCl (1.1 eq) and triethylamine (3 eq). The resulting reaction mixture is allowed to stir at about 100° C. for about 2 h to provide the chemical entity 4.

In a step for preparation of the chemical entity 8, for example, the chemical entity 6 (1 eq) and the chemical entity 7 (1.5 eq) are refluxed in benzene using a Dean-Stark apparatus for about 46 hours.

Utilizing 6-chloro-2H-[1,3]dioxolo[4,5-g]quinoline-7-carbonitrile of Scheme 1, the chemical entity of Formula II can be alternatively prepared in accordance with the chemical synthesis of Scheme 2.

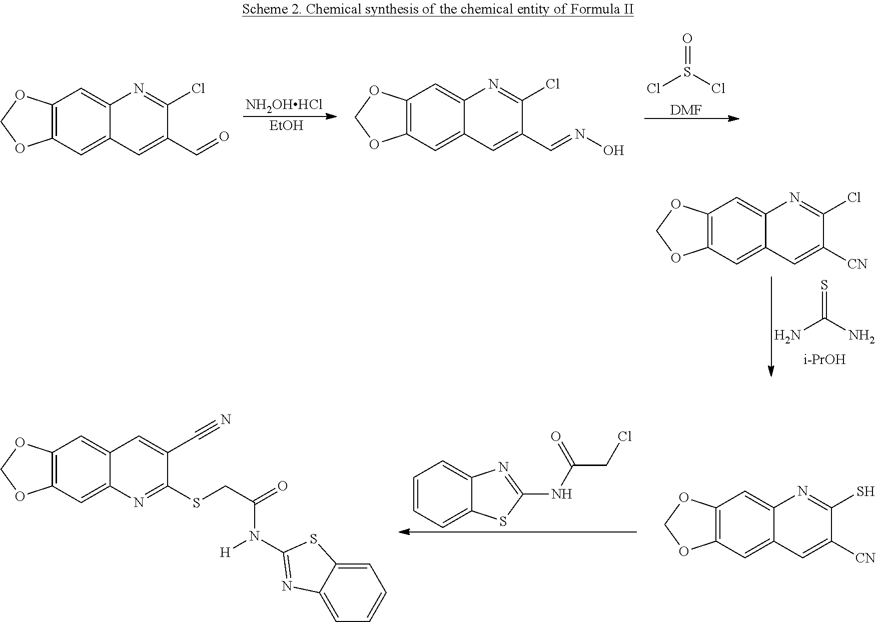

In view of Schemes 1 and 2, other chemical entities of Formula I such as those of at least Formulas IV-VII can be prepared in accordance with the chemical synthesis of Scheme 3.

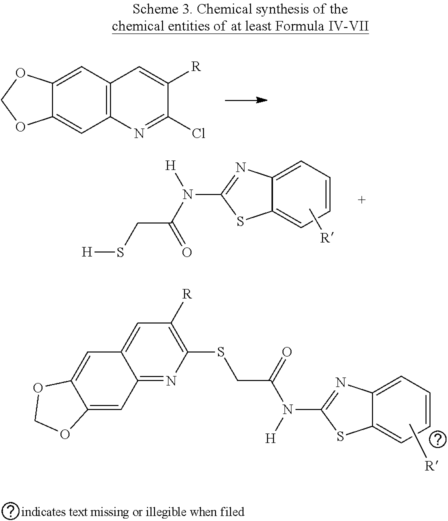

It should be understood Scheme 3 considers modifications to certain chemical entities, steps, or both of Schemes 1 or 2 for the preparation of the chemical entities of at least Formulas IV-VII.

The following eight chemical entities of Formula I can be prepared in accordance with that described in International Patent Application No. PCT/EP2014/063723, which published as WO 2014/207213 on Dec. 31, 2014.

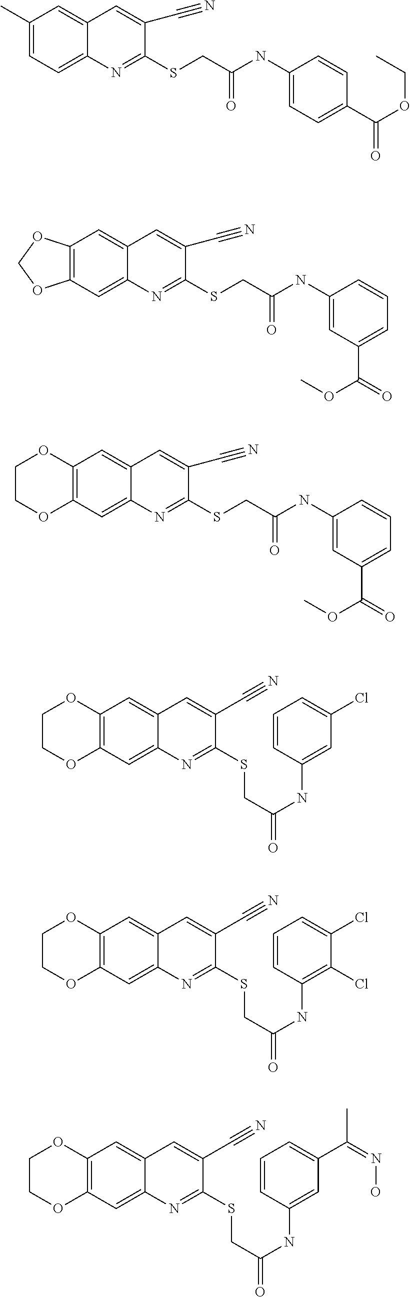 [SMILES: CCOC(=O)c1ccc(NC(=O)CSc2nc3ccc(C)cc3cc2C#N)cc1 ; COC(=O)c1cccc(NC(=O)CSc2nc3cc4c(cc3cc2C#N)OCO4)c1 ; COC(=O)c1cccc(NC(=O)CSc2nc3cc4c(cc3cc2C#N)OCCO4)c1 ; N#Cc1cc2cc3c(cc2nc1SCC(=O)Nc1cccc(Cl)c1)OCCO3 ; N#Cc1cc2cc3c(cc2nc1SCC(=O)Nc1cccc(Cl)c1Cl)OCCO3 ; C/C(=N/O)c1cccc(NC(=O)CSc2nc3cc4c(cc3cc2C#N)OCCO4)c1]

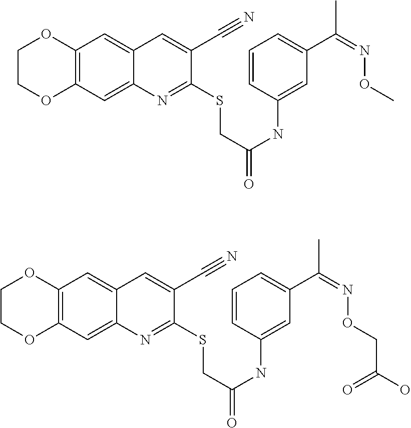 [SMILES: CO/N=C(/C)c1cccc(NC(=O)CSc2nc3cc4c(cc3cc2C#N)OCCO4)c1 ; C/C(=N/OCC(=O)O)c1cccc(NC(=O)CSc2nc3cc4c(cc3cc2C#N)OCCO4)c1]

### Pharmaceutical Formulations

The chemical entities disclosed herein can be formulated as pharmaceutical formulations for treating fibrosis with additives such as pharmaceutically acceptable excipients, pharmaceutically acceptable carriers, and pharmaceutically acceptable vehicles. Suitable pharmaceutically acceptable excipients, carriers, and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low-melting waxes, ion-exchange resins, or the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), each of which are incorporated herein by reference.

A pharmaceutical formulation can comprise a unit dose formulation, where the unit dose is a dose sufficient to have a therapeutic or suppressive effect or an amount effective to modulate or normalize a state of the disease. The unit dose may be sufficient as a single dose to have a therapeutic or suppressive effect or an amount effective to normalize the state of the disease. Alternatively, the unit dose may be a dose administered periodically in a course of treatment or suppression of the disease, or to modulate or normalize the state of the disease.

Pharmaceutical formulations containing the chemical entities disclosed herein can be in any form suitable for the intended method of administration, including, for example, a solution, a suspension, or an emulsion. Liquid carriers are typically used in preparing solutions, suspensions, or emulsions. Liquid carriers for use with the chemical entities disclosed herein include, for example, water, saline, pharmaceutically acceptable organic solvent(s), pharmaceutically acceptable oils or fats, or the like, as well as mixtures of two or more thereof. The liquid carrier may contain other pharmaceutically acceptable additives such as solubilizers, emulsifiers, nutrients, buffers, preservatives, suspending agents, thickening agents, viscosity regulators, stabilizers, or the like. Suitable organic solvents include, for example, monohydric alcohols, such as ethanol, or polyhydric alcohols, such as glycols. Suitable oils include, for example, soybean oil, coconut oil, olive oil, safflower oil, cottonseed oil, or the like. For parenteral administration, the carrier can also be an oily ester such as ethyl oleate, isopropyl myristate, or the like. Pharmaceutical formulations can also be in the form of microparticles, microcapsules, liposomal encapsulates, or the like, as well as combinations of any two or more thereof.

Time-release or controlled release delivery systems can be used, such as a diffusion-controlled matrix system or an erodible system, as described for example in: Lee, “Diffusion-Controlled Matrix Systems,” pp. 155-198 and Ron and Langer, “Erodible Systems”, pp. 199-224, in “Treatise on Controlled Drug Delivery,” A. Kydonieus Ed., Marcel Dekker, Inc., New York 1992. The matrix may be, for example, a biodegradable material that can degrade spontaneously in situ and in vivo for, example, by hydrolysis or enzymatic cleavage, for example, by proteases. The delivery system can be, for example, a naturally occurring or synthetic polymer or copolymer, for example, in the form of a hydrogel. Example polymers with cleavable linkages include polyesters, polyorthoesters, polyanhydrides, polysaccharides, poly(phosphoesters), polyamides, polyurethanes, poly(imidocarbonates), or poly(phosphazenes).

The chemical entities disclosed herein can be administered enterally, orally, parenterally, sublingually, by inhalation (e.g. as mists or sprays), rectally, or topically in dosage unit formulations containing conventional nontoxic pharmaceutically acceptable carriers, adjuvants, or vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, or the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The chemical entities are mixed with pharmaceutically acceptable carriers, adjuvants, and vehicles appropriate for the desired route of administration. Oral administration is a preferred route of administration, and formulations suitable for oral administration are preferred formulations. The chemical entities disclosed herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, or in other suitable forms. The chemical entities can also be administered in liposome formulations. The chemical entities can also be administered as prodrugs, where the prodrug undergoes transformation in the treated subject to a form which is therapeutically effective.

Where a formulation is used for injection or other parenteral administration including the routes listed herein, but also including embodiments used for oral, gastric, gastrointestinal, or enteric administration, the formulations and preparations used in the methods of the invention are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211).

Injectable preparations, for example, sterile injectable aqueous or oleaginous suspensions, are formulated using suitable dispersing or wetting agents and suspending agents. The sterile injectable preparation can also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are can be employed as a solvent or suspending medium. For this purpose, any bland fixed oil may be employed including synthetic mono- or diglycerides. In addition, fatty acids such as oleic acid find use in the preparation of injectables.

Solid dosage forms for oral administration can include capsules, tablets, pills, powders, and granules. In such solid dosage forms, a chemical entity ca be admixed with at least one inert diluent such as sucrose, lactose, or starch. Such dosage forms may also comprise additional substances other than inert diluents, for example, lubricating agents such as magnesium stearate. In the case of capsules, tablets, and pills, the dosage forms can also include buffering agents. Tablets and pills can additionally be prepared with enteric coatings.

Liquid dosage forms for oral administration may include pharmaceutically acceptable emulsions, solutions, suspensions, syrups, or elixirs containing inert diluents commonly used in the art such as water. Such pharmaceutical formulations can also comprise adjuvants, such as wetting agents, emulsifying and suspending agents, cyclodextrins, and sweetening, flavoring, or perfuming agents.

The chemical entities disclosed herein can also be administered in the form of liposomes. Liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. Pharmaceutical formulations in liposome form can contain, in addition to a chemical entity disclosed herein, stabilizers, preservatives, excipients, or the like. Preferred lipids are the phospholipids and phosphatidyl cholines (lecithins), both natural and synthetic. Methods to form liposomes are set forth in, for example, Prescott, Ed., Methods in Cell Biology, Volume XIV, Academic Press, New York, N.W., p. 33 et seq (1976), which is incorporated herein by reference.

The amount of a chemical entity disclosed herein that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific chemical entity employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount or effective amount for a given situation can be readily determined by routine experimentation and is within the skill and judgment of the ordinary clinician.

An example dosage that can be used is a therapeutically effective amount or effective amount within a dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Chemical entities disclosed herein can be administered in a single daily dose, or the total daily dosage may be administered in divided dosage of two, three or four times daily.

While the chemical entities disclosed herein can be administered as a sole active pharmaceutical agent, a combination with one or more other active agents can be used in the treatment or suppression of the disease. Representative agents useful in combination with the chemical entities disclosed herein for the treatment or suppression of the disease include, but are not limited to Pirfenidone or Nintedanib. When such additional active agents are used in combination with the chemical entities disclosed herein, the additional active agents can generally be employed in therapeutic amounts as indicated in the Physicians' Desk Reference (PDR) 53rd Edition (1999), or such therapeutically useful amounts as might be known to those of ordinary skill in the art.

The chemical entities disclosed herein and the other therapeutically active agents can be administered at maximum clinical dosage or at lower doses. Dosage levels of the chemical entities disclosed herein can be varied so as to obtain a desired therapeutic response depending on the route of administration, severity of the disease, or the response of the patient. When administered in combination with other therapeutic agents, the chemical entities disclosed herein can be formulated as separate pharmaceutical formulations that are given at the same time or different times, or the therapeutic agents can be given as a single pharmaceutical formulation.

A pharmaceutical formulation for treating fibrosis includes a chemical entity of Formula I, or a pharmaceutically acceptable salt thereof, and one or more pharmaceutically acceptable excipients, carriers, vehicles, or a combination thereof. In Formula I, R1 is selected from alkyl, alkenyl, alkynyl, cyano, halo, and azido; R2 is selected from arylamino, cycloalkylamino, heteroarylamino, and heterocycloalkylamino optionally substituted with alkyl, alkenyl, alkynyl, halo, acyl, alkoxycarbonyl, aminocarbonyl, or a combination thereof; R3 and R4 are either independently selected from alkoxy and heterocycloalkyl or together form O—CH₂—O or O—CH₂CH₂—O; and X is selected from S, O, (CH₂)ₙ, NH, and NR5; R5 is alkyl; and n is a positive integer from 1 to 5.

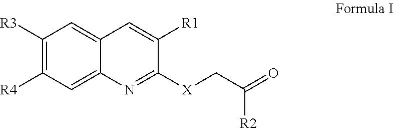

In some embodiments, R1 is selected from alkyl, alkenyl, alkynyl, cyano, halo, and azido; R2 is selected from heteroarylamino and heterocycloalkylamino optionally substituted as set forth above; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

In some embodiments, R1 is selected from methyl, ethyl, ethynyl, cyano, chloro, and azido; R2 is selected from 2-aminobenzothiazole and 2-aminobenzothiophene optionally substituted as set forth above at any one or more carbon atoms of carbon atoms 4, 5, 6, or 7 as conventionally numbered; R3 and R4 are both methoxy or together form O—CH₂—O; X is selected from S and (CH₂)ₙ; and n is a positive integer from 1 to 3.

As set forth above with respect to chemical entities for treating fibrosis, Formula I can be further defined, which can include, but is not limited to, any formula of Formulas II-IX.

In some embodiments, the pharmaceutical formulation is for treating pulmonary fibrosis including idiopathic pulmonary fibrosis or viral pneumonia-induced pulmonary fibrosis. The viral pneumonia can be caused by a virus selected from an adenovirus, a species of Metapneumovirus, a respiratory syndrome-related coronavirus, and any species of Orthohantamovirus. For example, the virus can be the respiratory syndrome-related coronavirus that causes COVID-19.

### Packaged Pharmaceutical Formulations

A packaged pharmaceutical formulation for treating fibrosis includes a pharmaceutical formulation including a chemical entity of Formula I, or a pharmaceutically acceptable salt thereof, and instructions for using the pharmaceutical formulation to treat a patient having fibrosis or susceptible to fibrosis. In Formula I, R1 is selected from alkyl, alkenyl, alkynyl, cyano, halo, and azido; R2 is selected from arylamino, cycloalkylamino, heteroarylamino, and heterocycloalkylamino optionally substituted with alkyl, alkenyl, alkynyl, halo, acyl, alkoxycarbonyl, aminocarbonyl, or a combination thereof; R3 and R4 are either independently selected from alkoxy and heterocycloalkyl or together form O—CH₂—O or O—CH₂CH₂—O; X is selected from S, O, (CH₂)ₙ, NH, and NR5; R5 is alkyl; and n is a positive integer from 1 to 5.

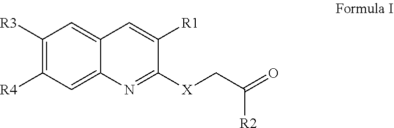

In some embodiments, R1 is selected from alkyl, alkenyl, alkynyl, cyano, halo, and azido; R2 is selected from heteroarylamino and heterocycloalkylamino optionally substituted as set forth above; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

In some embodiments, R1 is selected from methyl, ethyl, ethynyl, cyano, chloro, and azido; R2 is selected from 2-aminobenzothiazole and 2-aminobenzothiophene optionally substituted as set forth above at any one or more carbon atoms of carbon atoms 4, 5, 6, or 7 as conventionally numbered; R3 and R4 are both methoxy or together form O—CH₂—O; X is selected from S and (CH₂)ₙ; and n is a positive integer from 1 to 3.

As set forth above with respect to chemical entities for treating fibrosis, Formula I can be further defined, which can include, but is not limited to, any formula of Formulas II-IX.

In some embodiments, the packaged pharmaceutical formulation is for treating pulmonary fibrosis and the instructions are for using the pharmaceutical formulation to treat a patient having pulmonary fibrosis or susceptible to pulmonary fibrosis. In one example, the pharmaceutical formulation can be for treating idiopathic pulmonary fibrosis and the instructions can be for using the pharmaceutical formulation to treat a patient having idiopathic pulmonary fibrosis or susceptible to idiopathic pulmonary fibrosis. In another example, the pharmaceutical formulation can be for treating viral pneumonia-induced pulmonary fibrosis and the instructions can be for using the pharmaceutical formulation to treat a patient having viral pneumonia-induced pulmonary fibrosis or susceptible to viral pneumonia-induced pulmonary fibrosis. The viral pneumonia can be caused by a virus selected from an adenovirus, a species of Metapneumovirus, a respiratory syndrome-related coronavirus, and any species of Orthohantamovirus. Indeed, the virus can be the respiratory syndrome-related coronavirus that causes COVID-19.

### Methods for Treating Fibrosis

A method for treating fibrosis includes administering a pharmaceutical formulation including a therapeutically effective amount of a chemical entity of Formula I, or a pharmaceutically acceptable salt thereof. In Formula I, R1 is selected from alkyl, alkenyl, alkynyl, cyano, halo, and azido; R2 is selected from arylamino, cycloalkylamino, heteroarylamino, and heterocycloalkylamino optionally substituted with alkyl, alkenyl, alkynyl, halo, acyl, alkoxycarbonyl, aminocarbonyl, or a combination thereof; R3 and R4 are either independently selected from alkoxy and heterocycloalkyl or together form O—CH₂—O or O—CH₂CH₂—O; X is selected from S, O, (CH₂)ₙ, NH, and NR5; R5 is alkyl; and n is a positive integer from 1 to 5.

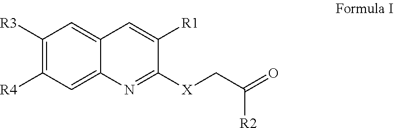

In some embodiments, R1 is selected from alkyl, alkenyl, alkynyl, cyano, halo, and azido; R2 is selected from heteroarylamino and heterocycloalkylamino optionally substituted as set forth above; R3 and R4 are either independently selected from alkoxy and heterocycloalkyl or together form O—CH₂—O; X is selected from S, O, (CH₂)ₙ, NH, and NR5; R5 is alkyl; and n is a positive integer from 1 to 5.

In some embodiments, R1 is selected from methyl, ethyl, ethynyl, cyano, chloro, and azido; R2 is selected from 2-aminobenzothiazole and 2-aminobenzothiophene optionally substituted as set forth above at any one or more carbon atoms of carbon atoms 4, 5, 6, or 7 as conventionally numbered; R3 and R4 are both methoxy or together form O—CH₂—O; X is selected from S and (CH₂)ₙ; and n is a positive integer from 1 to 3.

As set forth above with respect to chemical entities for treating fibrosis, Formula I can be further defined, which can include, but is not limited to, any formula of Formulas II-IX.

In some embodiments, the method is for treating pulmonary fibrosis including idiopathic pulmonary fibrosis or viral pneumonia-induced pulmonary fibrosis. The viral pneumonia can be caused by a virus selected from an adenovirus, a species of Metapneumovirus, a respiratory syndrome-related coronavirus, and any species of Orthohantamovirus. For example, the virus can be the respiratory syndrome-related coronavirus that causes COVID-19.

### In Vitro Model and Drug Screen for Fibrosis

To address the shortcomings of the current in vitro and in vivo models for fibrosis, a relatively rapid 96 well-based assay was employed to identify chemical entities that would be likely to show clinical efficacy by better mimicking clinical fibrotic diseases. To this end, induced pluripotent stem cells (iPSCs) from familial IPF patients were used for an in vitro model of progressive fibrosis. The assay exhibits the hallmarks of the clinical fibrotic disease state: progressive collagen deposition, cell stiffness, cytokine and chemokine production, and progressive activation of TGF-β. Using this assay, chemical entities have been identified with the ability to prevent and reverse the signs of fibrosis in this assay, as well as in IPF fibrotic lung slice cultures from patients with end-stage lung disease. In fact, a reduction in collagen and α-smooth muscle actin (α-SMA) was seen, as well as a reversal of chronic inflammation in the IPF samples in the assay. These effects are the critical components of progressive fibrosis and are essential in order to reverse clinical fibrosis. Finally, the chemical entities were also effective in the in vivo bleomycin model in aged mice, which unlike young mice develop persistent fibrotic changes in their lungs.

A human in vitro model was also employed that closely phenocopies IPF. To achieve this, iPSCs from lung biopsies of familial IPF patients were generated and then differentiated along epithelial, mesenchymal, and immune cell lineages. These cells, when cultured on relatively stiff (13 kPa) hydrogels, were able to interact and drive the spontaneous and progressive formation of aggregates of cells that behave as activated fibroblasts or myofibroblasts, as confirmed by expression of α-SMA and vimentin, markers of myofibroblasts and mesenchymal cells, respectively. Increased collagen1 and α-SMA were also seen by immunoblotting. These cellular aggregates lay down extracellular matrix, such as collagen1, in a progressive manner. The Plasminogen Activator Inhibitor (PAI-1) promoter is activated in the presence of TGF-β. Mink lung epithelial cells were cultured that stably express luciferase under the control of the PAI-1 promoter and examined the effect of conditioned media from our model on the cells. The luciferase results demonstrated that the model is actively producing increasing amounts of TGF-β over time in culture. In the in vitro hydrogel model, many cells within the fibrotic foci were proliferative, as assessed by EdU incorporation, which is one of the features of the fibroblasts or myofibroblasts seen in IPF. Fifteen iPSC cell lines derived from five IPF patients have been examined, and they all develop the fibrotic foci phenotype in the in vitro culture system, demonstrating the reproducibility of the model.

### Screening Protocol

Primary IPF 96-well efficacy screen: Efficacy cut off: Greater than 80% inhibition of the fibrotic phenotype, which includes the size and intensity of the induced fibroblast activation, shape of cells, and cellular viability in the presence of a viability dye (Calcein AM) with an EC₅₀≤100 nM.

Secondary IPF 96-well efficacy screen: Greater than 80% reversal of fibrotic phenotype with an IC₅₀ at ≤100 nM.

Toxicity screen: An IC₅₀ of ≥1,000 nM is needed to ensure the chemical entity is not toxic and not cytostatic.

Ex vivo IFP lung slice screen: At least 50% reduction in collagen and α-SMA gene expression with p-value <0.05 at a concentration that is ≤5-fold greater than the IC₅₀ defined above.

Solubility screen: ≥50 mg/mL aqueous solution is ideal, but a lower solubility is tolerable depending on the solvent. All chemical entities tested for solubility that passed the primary screen.

Reduction of IC₅₀s to 100 nM or less demonstrates an ability to dial in improved IC₅₀s. While not optimal, a drug with an IC₅₀ of 100 nM in a whole cell assay is still appropriate for taking to the clinic if other key drug properties are appropriate. Other such drug properties can be investigated. Likewise, a 10-fold in vitro toxicity separation is appropriate as a starting point for further development. Drugs are usually less potent when tested in slice models for many well-known reasons. Thus, a 5-fold shift was chosen. The slice model was carried out at five concentrations.

Briefly for the primary and secondary 96-well fibrosis efficacy model, the cells were imaged with phase contrast imaging at 7-10 days. The live cell dye (Calcein AM) was added just before imaging at a concentration of 0.5 μg/mL. The fluorescence signal was found to be stable for at least 3 hours after addition of the Calcein AM. A concentration range of 50 nM to 50 μM in 10-fold increments was tested per well in triplicate using DMSO as a negative control. Concentration ranges were changed in accordance with potency. Dose response curves were be plotted against the number of fibrotic aggregates to calculate the IC₅₀ for each analog. Counter screening with CellTitreGlo ensured the chemical entities were not effective through cytostasis.

Testing in the ex vivo IPF lung tissue and tissues from other fibrotic organs can be used to confirm the most promising chemical entities, as this is a cost-effective model that closely represents the targeted disease. For the ex vivo IPF lung tissue slice model, IPF tissue was obtained from patients with end-stage lung disease at the time of their lung transplant. All IPF tissue, regardless of gender, race, or ethnicity, were assessed for efficacy with the chemical entities. In addition, the efficacy in reversing fibrosis in the ex vivo model after 72 hours of treatment with the chemical entities was compared to DMSO, Pirfenidone, and Nintedanib control treatments, as well as combinations with Pirfenidone or Nintedanib. The treated IPF tissue was assessed with the hydroxyproline assay and picosirius red and Wright stains. Tissue remodeling with immunostaining for epithelial and mesenchymal markers was also examined, including EPCAM, SSEA4, and PDGF-α.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures can be made from the particular embodiments disclosed herein without departing from the scope of the concepts provided herein.

What is claimed is:

1. A chemical entity of Formula I 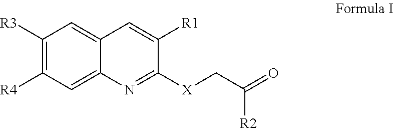
or a pharmaceutically acceptable salt thereof, wherein
R1 is selected from alkyl, alkenyl, alkynyl, cyano, halo, and azido;
R2 is selected from heteroarylamino and heterocycloalkylamino;
R3 and R4 are either independently selected from alkoxy and heterocycloalkyl or together form O—CH₂—O;
X is selected from S, O, (CH₂)ₙ, NH, and NR5;
R5 is alkyl; and
n is a positive integer from 1 to 5.

2. The chemical entity or salt of claim 1, wherein
R1 is selected from methyl, ethyl, ethynyl, cyano, chloro, and azido;
R2 is selected from 2-aminobenzothiazole and 2-aminobenzothiophene;
R3 and R4 are both methoxy or together form O—CH₂—O;
X is selected from S and (CH₂)ₙ; and
n is a positive integer from 1 to 3.

3. The chemical entity or salt of claim 1, wherein R1 is cyano, R2 is 2-aminobenzothiazole, R3 and R4 together form O—CH₂—O, and X is S, thereby providing the chemical entity of Formula II.

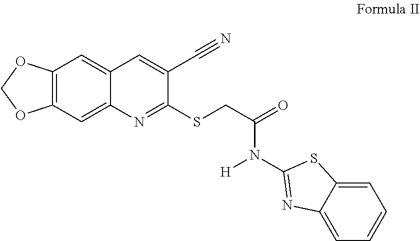 [SMILES: N#Cc1cc2cc3c(cc2nc1SCC(=O)Nc1nc2ccccc2s1)OCO3]

4. The chemical entity or salt of claim 1, wherein R1 is ethynyl, R2 is 2-aminobenzothiophene, R3 and R4 together form O—CH₂—O, and X is S, thereby providing the chemical entity of Formula III.

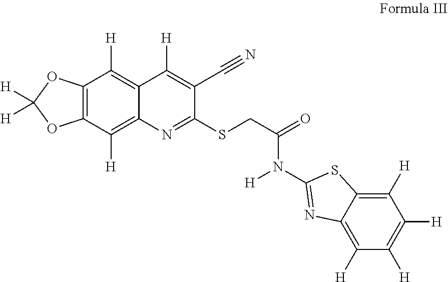 [SMILES: N#Cc1cc2cc3c(cc2nc1SCC(=O)Nc1nc2ccccc2s1)OCO3]

5. The chemical entity or salt of claim 1, wherein R1 is ethynyl, R2 is 2-aminobenzothiazole, R3 and R4 together form O—CH₂—O, and X is S, thereby providing the chemical entity of Formula IV.

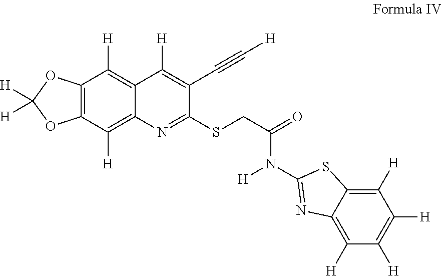 [SMILES: C#Cc1cc2cc3c(cc2nc1SCC(=O)Nc1nc2ccccc2s1)OCO3]

6. The chemical entity or salt of claim 1, wherein R1 is methyl, R2 is 2-aminobenzothiazole, R3 and R4 together form O—CH₂—O, and X is S, thereby providing the chemical entity of Formula V.

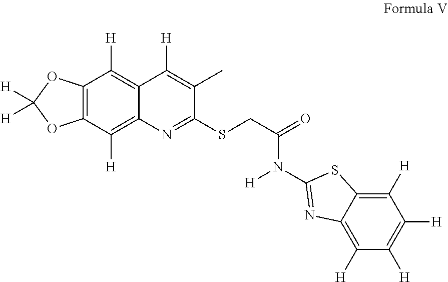 [SMILES: Cc1cc2cc3c(cc2nc1SCC(=O)Nc1nc2ccccc2s1)OCO3]

7. The chemical entity or salt of claim 1, wherein R1 is ethyl, R2 is 2-aminobenzothiazole, R3 and R4 together form O—CH₂—O, and X is S, thereby providing the chemical entity of Formula VI.

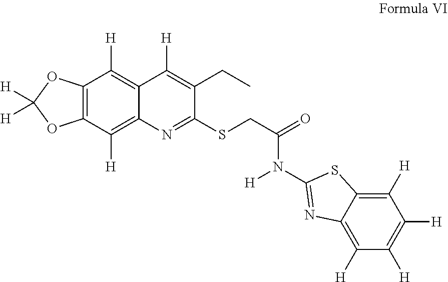 [SMILES: CCc1cc2cc3c(cc2nc1SCC(=O)Nc1nc2ccccc2s1)OCO3]

8. The chemical entity or salt of claim 1, wherein R1 is chloro, R2 is 2-aminobenzothiazole, R3 and R4 together form O—CH₂—O, and X is S, thereby providing the chemical entity of Formula VII.

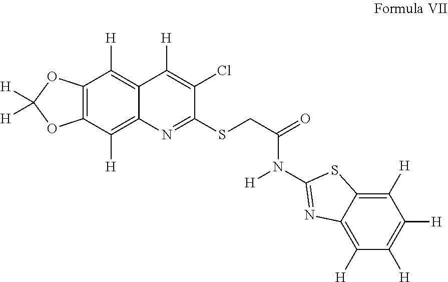 [SMILES: O=C(CSc1nc2cc3c(cc2cc1Cl)OCO3)Nc1nc2ccccc2s1]

9. The chemical entity or salt of claim 1, wherein R1 is cyano, R2 is 2-aminobenzothiazole, R3 and R4 together form O—CH₂—O, X is (CH₂)ₙ, and n is 3, thereby providing the chemical entity of Formula VIII.

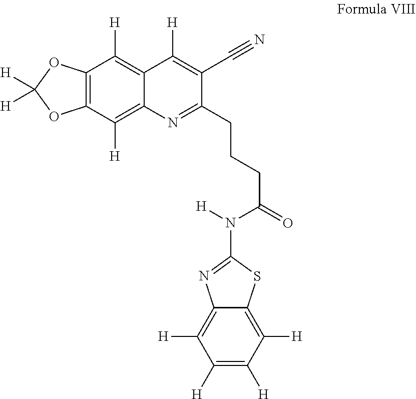 [SMILES: N#Cc1cc2cc3c(cc2nc1CCCC(=O)Nc1nc2ccccc2s1)OCO3]

10. The chemical entity or salt of claim 1, wherein R1 is azido, R2 is 2-aminobenzothiazole, R3 and R4 together form O—CH₂—O, X is (CH₂)ₙ, and n is 3, thereby providing the chemical entity of Formula IX.

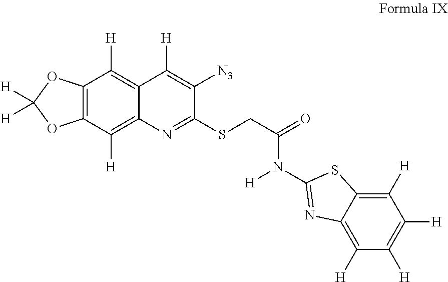 [SMILES: [N-]=[N+]=Nc1cc2cc3c(cc2nc1SCC(=O)Nc1nc2ccccc2s1)OCO3]

11. A pharmaceutical formulation for treating fibrosis, comprising:
a) a chemical entity of Formula I 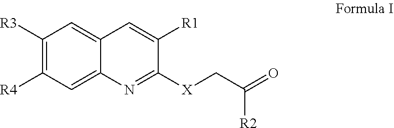
or a pharmaceutically acceptable salt thereof, wherein
R1 is selected from alkyl, alkenyl, alkynyl, cyano, halo, and azido;
R2 is selected from heteroarylamino and heterocycloalkylamino;
R3 and R4 are either independently selected from alkoxy and heterocycloalkyl or together form O—CH₂—O;
X is selected from S, O, (CH₂)ₙ, NH, and NR5;
R5 is alkyl; and
n is a positive integer from 1 to 5; and
b) one or more pharmaceutically acceptable excipients, carriers, vehicles, or a combination thereof.

12. The pharmaceutical formulation of claim 11, wherein the pharmaceutical formulation is for treating pulmonary fibrosis.

13. The pharmaceutical formulation of claim 11, wherein the pharmaceutical formulation is for treating idiopathic pulmonary fibrosis.

14. The pharmaceutical formulation of claim 11, wherein the pharmaceutical formulation is for treating viral pneumonia-induced pulmonary fibrosis.

15. The pharmaceutical formulation of claim 14, wherein the viral pneumonia is caused by a virus selected from an adenovirus, a species of Metapneumovirus, a respiratory syndrome-related coronavirus, and any species of Orthohantamovirus.

16. The pharmaceutical formulation of claim 15, wherein the virus is the respiratory syndrome-related coronavirus that causes coronavirus disease 2019 (COVID-19).

17. The pharmaceutical formulation of claim 11, wherein the pharmaceutical formulation is formulated for a mode of administration selected from oral, subcutaneous, transdermal, transmucosal, iontophoretic, intravenous, intraarterial, intramuscular, intraperitoneal, intranasal, subdural, rectal, gastrointestinal, or directly to a specific or affected tissue or organ.

18. A packaged pharmaceutical formulation for treating fibrosis, comprising:
a) a pharmaceutical formulation including a chemical entity of Formula I 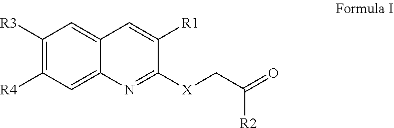
or a pharmaceutically acceptable salt thereof, wherein
R1 is selected from alkyl, alkenyl, alkynyl, cyano, halo, and azido;
R2 is selected from heteroarylamino and heterocycloalkylamino;
R3 and R4 are either independently selected from alkoxy and heterocycloalkyl or together form O—CH₂—O;
X is selected from S, O, (CH₂)ₙ, NH, and NR5;
R5 is alkyl; and
n is a positive integer from 1 to 5; and
b) instructions for using the pharmaceutical formulation to treat a patient having idiopathic pulmonary fibrosis or susceptible to idiopathic pulmonary fibrosis.

19. The packaged pharmaceutical formulation of claim 18, wherein the pharmaceutical formulation is for treating pulmonary fibrosis and the instructions are for using the pharmaceutical formulation to treat a patient having pulmonary fibrosis or susceptible to pulmonary fibrosis.

20. The packaged pharmaceutical formulation of claim 18, wherein the pharmaceutical formulation is for treating idiopathic pulmonary fibrosis and the instructions are for using the pharmaceutical formulation to treat a patient having idiopathic pulmonary fibrosis or susceptible to idiopathic pulmonary fibrosis.

21. The packaged pharmaceutical formulation of claim 18, wherein the pharmaceutical formulation is for treating viral pneumonia-induced pulmonary fibrosis and the instructions are for using the pharmaceutical formulation to treat a patient having viral pneumonia-induced pulmonary fibrosis or susceptible to viral pneumonia-induced pulmonary fibrosis.

22. A method for treating fibrosis, comprising:
administering a pharmaceutical formulation including a therapeutically effective amount of a chemical entity of Formula I 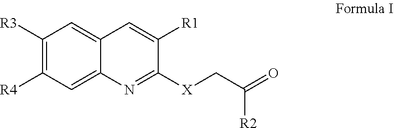
or a pharmaceutically acceptable salt thereof, wherein
R1 is selected from alkyl, alkenyl, alkynyl, cyano, halo, and azido;
R2 is selected from heteroarylamino and heterocycloalkylamino;
R3 and R4 are either independently selected from alkoxy and heterocycloalkyl or together form O—CH₂—O;
X is selected from S, O, (CH₂)ₙ, NH, and NR5;
R5 is alkyl; and
n is a positive integer from 1 to 5.

23. The method of claim 22, wherein the fibrosis is pulmonary fibrosis.

24. The method of claim 22, wherein the fibrosis is idiopathic pulmonary fibrosis.

25. The method of claim 22, wherein the fibrosis is viral pneumonia-induced pulmonary fibrosis.
